(19) United States
(12) Patent Application Publication
(10) Pub. No.: US 2017/0072096 A1
(43) Pub. Date: Mar. 16, 2017

(54) NOVEL HYDROGELS AND METHODS USING SAME

(71) Applicant: DREXEL UNIVERSITY, Philadelphia, PA (US)

(72) Inventors: Lynn S. PENN, Philadelphia, PA (US); Xiaohe XU, Cherry Hill, NJ (US); Xue SHA, Waukesha, WI (US); Michele MARCOLONGO, Aston, PA (US); Caroline SCHAUER, Hulmeville, PA (US); Katsiaryna PRUDNIKOVA, Huntingdon Valley, PA (US)

(21) Appl. No.: 15/120,824

(22) PCT Filed: Mar. 16, 2015
(86) PCT No.: PCT/US15/20720

§ 371 Date: Aug. 23, 2016

Related U.S. Application Data
(60) Provisional application No. 61/953,865, filed on Mar. 16, 2014.

(51) Int. Cl. A61L 27/20; A61L 27/52; C08J 3/075

(52) U.S. Cl. CPC A61L 27/20; C08J 3/075; A61L 27/52; C08J 2305/08; A61L 2430/38

(57) ABSTRACT

The present invention provides hydrogel-based compositions that are suitable for replacing or supplementing the nucleus pulposus in a subject. The compositions of the invention are useful for treating, ameliorating or reverting degradation of the nucleus pulposus in the subject. The present invention also provides methods of preparing and using such compositions.

### CROSS-REFERENCE TO RELATED APPLICATIONS

The present application claims priority under 35 U.S.C. §119 (e) to U.S. Provisional Application No. 61/953,865, filed Mar. 16, 2014, which is hereby incorporated by reference in its entirety herein.

### BACKGROUND OF THE INVENTION

Nucleus pulposus is a gelatinous material in the center of the intervertebral disc, and it is bound circumferentially by the annulus fibrosus and is confined at top and bottom by the vertebral end plates (Raj, 2008, Pain Practice 8 (1):18-44). This constraint puts the nucleus pulposus in a state of hydrostatic stress. The nucleus pulposus functions to distribute hydraulic pressure in all directions within each disc under compressive loads.

Autopsy studies have shown that the nucleus pulposus is composed of water (70-90% by weight), proteoglycan aggrecans (14% by weight) and collagen (4% by weight). Attached to each aggrecan molecule are the glycosaminoglycan (GAG) chains of chondroitin sulfate (a sulfated polysaccharide) and keratin sulfate. Aggrecan is negatively charged, allowing the nucleus pulposus to attract water molecules. With aging, repetitive loading or a significant overload, the nucleus pulposus degrades, losing water content and size, and probably losing material other than water. With the degradation of the nucleus pulposus, the load on the spine cannot be distributed evenly or uniformly. The severity of clinically observable disc degeneration varies widely from bulging, herniated and ruptured discs to advanced spondylosis leading to spinal stenosis, spondylolithesis and scoliosis. Patients suffering from a degenerated disc may experience a number of symptoms, including pain of the lower back, buttocks and legs, and sciatica (Adams, 2004, Acup. Med. 22 (4):178).

Efforts to prevent or reverse the degradation of the nucleus pulposus are hampered by the fact that this structure is not well characterized. In fact, up to recently there was much debate whether the nucleus pulposus was a liquid or a viscoelastic solid. Since the nucleus pulposus is confined volumetrically, either state would provide for equalization of hydrostatic stress generated by external forces. Further, it is difficult to obtain representative specimens of the nucleus pulposus. The main source of samples is autopsy procedures, which are generally not correlated with state of spine degeneration at the time of death. Thus, nucleus pulposus specimens are not only rare but also suffer from great inherent variability. Reproducing the mechanical properties of the nucleus pulposus is thought to be a key requirement for a proposed synthetic replacement or supplement of this structure.

There is a need in the art for the development of a novel material that can be used to replace or supplement nucleus pulposus in a subject. Such material should be biocompatible and capable of mimicking the natural properties of the nucleus pulposus. The present invention addresses this unmet need in the art.

### BRIEF SUMMARY OF THE INVENTION

The invention includes a composition comprising a polymerizable crosslinker and a scaffold. The invention further includes a method of preparing a hydrogel. The invention further includes a method of replacing or supplementing the nucleus pulposus in a subject in need thereof.

In certain embodiments, the polymerizable crosslinker comprises an oxiranyl group and an alkenyl group. In other embodiments, the scaffold comprises at least one selected from the group consisting of collagen, pectin, carrageenan, poly(L-lysine), gelatin, agarose, dextran sulfate, heparin, polygalacturonic acid, mucin, chondroitin sulfate, hyaluronic acid, chitosan, alginate, alginate sulfate, poly(acrylic acid), poly(methyl methacrylate) (PMMA), poly(vinylsulfonic acid) (PVSA), poly(L-aspartic acid), poly(L-glutamic acid), poly(L-aspartic acid)-grafted-poly(ethylene glycol) (PAA-g-PEG), poly(L-glutamic acid)-grafted-poly(ethylene glycol) (PGA-g-PEG), poly(sodium 4-styrenesulfonate) (PSS), dermatan sulfate, carboxymethyl cellulose (CMC), and any combinations thereof.

In certain embodiments, the scaffold comprises chondroitin sulfate. In other embodiments, the crosslinker comprises a glycidyl group. In yet other embodiments, the crosslinker comprises at least one selected from the group consisting of a glycidyl ester, amide, amine, ether, thioester, sulfonamide, and any combinations thereof. In yet other embodiments, the crosslinker comprises at least one selected from the group consisting of glycidyl methacrylate (GMA), glycidyl acrylate, allyl glycidyl ether, and any combinations thereof. In yet other embodiments, the polymerizable crosslinker comprises glycidyl methacrylate.

In certain embodiments, the scaffold and polymerizable crosslinker react to form a derivatized scaffold. In other embodiments, the ratio between the scaffold and the polymerizable crosslinker is selected so that at least partial crosslinking of the derivatized scaffold generates a hydrogel.

In certain embodiments, the composition further comprises a polymerization initiator comprising a chemical agent. In other embodiments, the chemical agent comprises at least one selected from the group consisting of an inorganic peroxide, azo compound, organic peroxide, and any combinations thereof. In yet other embodiments, the polymerization initiator is at least partially soluble in the composition. In yet other embodiments, at least a portion of the derivatized scaffold is crosslinked. In yet other embodiments, crosslinking of the scaffold is promoted by contacting the derivatized scaffold with a polymerization initiator comprising a chemical agent.

In certain embodiments, the composition comprises a hydrogel. In other embodiments, the hydrogel has about the same mechanical properties of the nucleus pulposus of a subject. In yet other embodiments, the hydrogel is suitable for implantation or transplantation into the subject. In yet other embodiments, the hydrogel is suitable for replacing or supplementing the nucleus pulposus in the subject. In yet other embodiments, the hydrogel further comprises cells. In yet other embodiments, the hydrogel is administered to the subject as a partially or totally dried gel.

In certain embodiments, the subject is a mammal In other embodiments, the mammal is a human. In yet other embodiments, the composition further comprises cells.

In certain embodiments, the method comprises contacting a scaffold and a polymerizable crosslinker to generate a derivatized scaffold. In other embodiments, the method comprises promoting at least partial crosslinking of the derivatized scaffold, whereby a hydrogel is formed. In yet other embodiments, the method comprises administering to the subject a therapeutically effective amount of any hydrogel of the present invention.

In certain embodiments, promoting at least partial crosslinking of the derivatized scaffold comprises contacting the derivatized scaffold with a polymerization initiator comprising a chemical agent.

### BRIEF DESCRIPTION OF THE DRAWINGS

The following detailed description of certain embodiments of the invention will be better understood when read in conjunction with the appended drawings. For the purpose of illustrating the invention, illustrated in the drawings are certain embodiments. It should be understood, however, that the invention is not limited to the precise arrangements and instrumentalities of the embodiments illustrated in the drawings.

FIGS. 1A-1B illustrate the nucleus pulposus. FIG. 1A is a schematic representation of the nucleus pulposus (reproduced from “The Nucleus of the Intervertebral Disc from Development to Degeneration,” by J. P. G. Urban, S. Roberts, and J. R. Ralphs, in American Zoologist 2000, vol. 40, pp. 53-61), and FIG. 1B illustrates progressive degeneration of the nucleus pulposus (top to bottom). In this series of images, the degradation is manifest as a shrinkage of the nucleus pulposus, which is located in the center of each image.

FIG. 2 illustrates the structure of chondroitin sulfate (CS).

FIG. 3 illustrates the structure of glycidyl methacrylate (GMA).

FIG. 4 is a table illustrating hydrogel formulations and water absorption capabilities for hydrogels of the invention.

FIG. 5 is a graph illustrating water absorption as a function of time for hydrogels of the invention.

FIG. 6 is a graph illustrating water loss as a function of time for hydrogels of the invention.

FIG. 7 shows the instrument configuration for applying various modes of deformation in dynamic mechanical analysis experiments. At bottom is a diagram of the configuration used for compression mode as applied to a cylindrical specimen.

FIG. 8 is a photographic image of the total instrument used for dynamic mechanical analysis, including, in non-limiting embodiments, load applicator, computer controller, and data read-out screen.

FIG. 9 is a graph illustrating complex compression modulus of hydrogels of the invention. CS to GMA ratios are indicated in legend box. Shaded area represents range of values (including standard deviations) reported for the human nucleus pulposus.

FIG. 10 is a graph illustrating storage (E′ ) and loss (E″ ) moduli for hydrogels of the invention. The legend shows the molar ratio of CS to GMA for each hydrogel.

FIG. 11 is a graph illustrating phase angle δ as a function of frequency for hydrogels of the invention.

FIG. 12 is a photograph showing cylindrical specimens of hydrogels of the invention, which are standing freely and are about 14 mm in diameter.

FIG. 13 is a table illustrating the effect of reaction time on composition and gel properties for hydrogels of the invention, for initial molar ratio CS : GMA of 1:200.

FIG. 14 is a schematic representation of the preparation of a non-limiting example of a hydrogel of the invention.

### DETAILED DESCRIPTION OF THE INVENTION

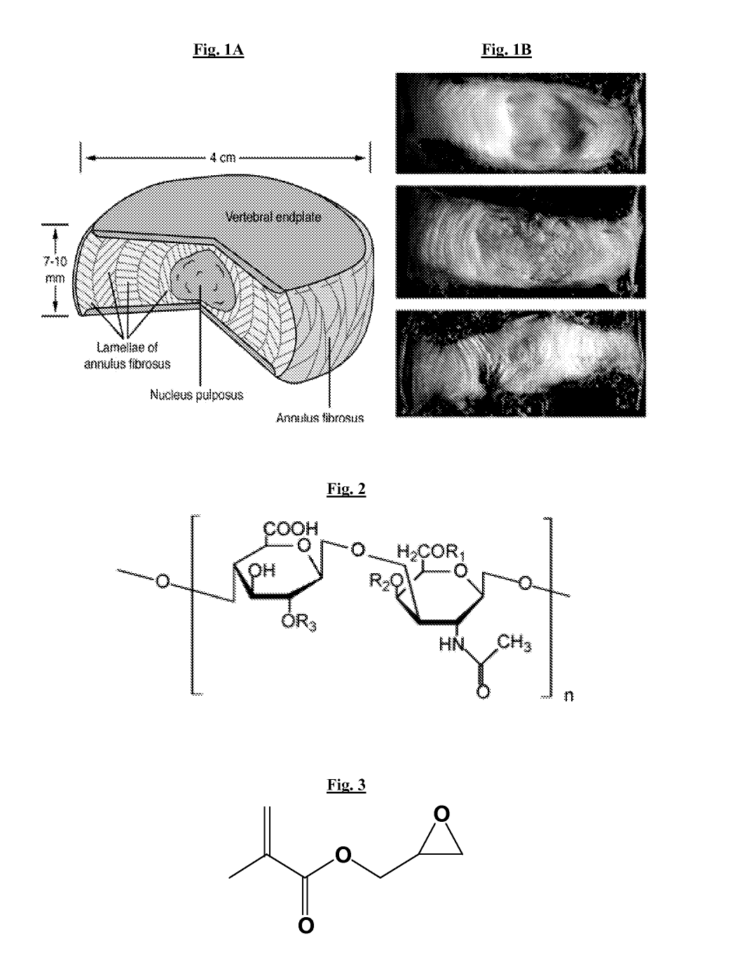

The present invention relates to the unexpected discovery of novel compositions that may be used to replace or supplement nucleus pulposus (FIGS. 1A-1B) in a subject. In one aspect, the compositions of the invention have tunable properties that may be adjusted to match the nucleus pulposus that needs to be replaced or supplemented.

In one aspect, the compositions of the invention comprise a hydrogel prepared by placing in contact a scaffold, such as chondroitin sulfate (CS), and a polymerizable crosslinker, such as glycidyl methacrylate (GMA), to generate a derivatized scaffold, and promoting at least partial polymerization (cross-linking) of the derivatized scaffold. In certain embodiments, the polymerizable crosslinker comprises an oxiranyl group (a strained ring) and an alkenyl group.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

As used herein, the term “abnormal” when used in the context of organisms, tissues, cells or components thereof, refers to those organisms, tissues, cells or components thereof that differ in at least one observable or detectable characteristic (e.g., age, treatment, or time of day) from those organisms, tissues, cells or components thereof that display the “normal” (expected) respective characteristic. Characteristics that are normal or expected for one cell or tissue type might be abnormal for a different cell or tissue type.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

As used herein, a disease or disorder is “alleviated” if the severity of a symptom of the disease or disorder, the frequency with which such a symptom is experienced by a patient, or both, is reduced.

The term “biodegradable” includes polymers, compositions and formulations, such as those described herein, that are intended to degrade during use. Biodegradable polymers typically differ from non-biodegradable polymers in that the former may be degraded during use. In certain embodiments, such use involves in vivo use, such as in vivo therapy. In other embodiments, such use involves in vitro use. In general, biodegradation involves the degradation of a biodegradable polymer into its component subunits, or digestion, e.g., by a biochemical process, of the polymer into smaller, non-polymeric subunits. Two types of biodegradation may generally be identified. For example, biodegradation may involve cleavage of bonds (whether covalent or otherwise) in the polymer backbone. In such biodegradation, monomers and oligomers typically result, and even more typically, such biodegradation occurs by cleavage of a bond connecting one or more of subunits of a polymer. Further, biodegradation may involve cleavage of a bond (whether covalent or otherwise) internal to a side chain or that connects a side chain to the polymer backbone. For example, a therapeutic agent or other chemical moiety attached as a side chain to the polymer backbone may be released by biodegradation. In certain embodiments, at least one type of biodegradation may occur during use of a polymer. As used herein, the term “biodegradation” encompasses all known types of biodegradation.

As used herein, the terms “biocompatible polymer” and “biocompatibility” when used in relation to polymers are recognized in the art. For example, biocompatible polymers include polymers that are generally neither toxic to the host, nor degrade (if the polymer degrades) at a rate that produces monomeric or oligomeric subunits or other byproducts at toxic concentrations in the host. In certain embodiments, biodegradation generally involves degradation of the polymer in a host, e.g., into its monomeric subunits, which may be known to be effectively non-toxic. Intermediate oligomeric products resulting from such degradation may have different toxicological properties, however, or biodegradation may involve oxidation or other biochemical reactions that generate molecules other than monomeric subunits of the polymer. Consequently, in certain embodiments, toxicology of a biodegradable polymer intended for in vivo use, such as implantation or injection into a patient, may be determined after one or more toxicity analyses. It is not necessary that any subject composition have a purity of 100% to be deemed biocompatible; indeed, it is only necessary that the subject compositions be biocompatible as set forth above. Hence, a subject composition may comprise polymers comprising 99%, 98%, 97%, 96%, 95%, 90%, 85%, 80%, 75% or even less of biocompatible polymers, e.g., including polymers and other materials and excipients described herein, and still be biocompatible.

As used herein, the term “CS” refers to chondroitin sulfate.

As used herein, a “disease” is a state of health of an animal wherein the animal cannot maintain homeostasis, and wherein if the disease is not ameliorated then the animal's health continues to deteriorate.

As used herein, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the term “GMA” refers to glycidyl methacrylate.

As used herein, the term “HA” refers to hyaluronic acid.

As used herein, the term “hydrogel” or “aquagel” refers to a network of water-soluble oligomers or polymer chains that are rendered insoluble by means of crosslinking, sometimes found as a colloidal gel in which water is the dispersion medium.

As used herein, an “instructional material” includes a publication, a recording, a diagram, or any other medium of expression that can be used to communicate the usefulness of a composition or method of the invention in the kit for treating, preventing or alleviating various diseases or disorders recited herein. The instructional material of the kit of the invention can, for example, be affixed to a container that contains the identified composition or delivery system of the invention or be shipped together with a container that contains the identified composition or delivery system. Alternatively, the instructional material can be shipped separately from the container with the intention that the instructional material and the composition be used cooperatively by the recipient.

As used herein, the terms “patient,” “subject,” “individual” and the like are used interchangeably, and refer to any animal, or organs, tissues or cells thereof whether in vitro or in situ, amenable to the methods described herein. In certain embodiments, the patient, subject or individual is a vertebrate. In other embodiments, the patient, subject or individual is a mammal In yet other embodiments, the patient, subject or individual is a human.

As used herein, the term “scaffold” refers to a polymeric material that can be derivatized by reaction with a crosslinker, such as a crosslinker comprising a oxiranyl group.

As used herein, the term “therapeutic” treatment refers to a treatment administered to a subject who exhibits signs of pathology, for the purpose of diminishing or eliminating those signs.

As used herein, the term “therapeutically effective amount” refers to an amount that is sufficient or effective to prevent or treat (delay or prevent the onset of, prevent the progression of, inhibit, decrease or reverse) a disease or condition described or contemplated herein, including alleviating symptoms of such disease or condition.

As used herein, the term “treating a disease or disorder” means reducing the frequency with which a symptom of the disease or disorder is experienced by a patient. Disease and disorder are used interchangeably herein.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 and the like, as well as individual numbers within that range, for example, 1, 2, 2.7, 3, 4, 5, 5.3, and 6. This applies regardless of the breadth of the range.

### Description

The present invention relates to the unexpected discovery of novel compositions that may be used to replace or supplement nucleus pulposus in a subject. In one aspect, the compositions of the invention have tunable properties that may be adjusted to match the nucleus pulposus that needs to be replaced or supplemented.

In one aspect, the compositions of the invention comprise a hydrogel prepared by contacting a scaffold and a polymerizable crosslinker to generate a derivatized scaffold, and promoting at least partial crosslinking of the derivatized scaffold. In certain embodiments, the polymerizable crosslinker comprises an oxiranyl group and an alkenyl group.

In certain embodiments, a scaffold is useful within the present invention. Non-limiting examples of scaffolds contemplated within the invention include but are not limited to, collagen, pectin, carrageenan, poly(L-lysine), gelatin, agarose, dextran sulfate, heparin, polygalacturonic acid, mucin, chondroitin sulfate, hyaluronic acid, chitosan, alginate, alginate sulfate, poly(acrylic acid), poly(methyl methacrylate) (PMMA), poly(vinylsulfonic acid) (PVSA), poly(L-aspartic acid), poly(L-glutamic acid), poly(L-aspartic acid)-grafted-polyethylene glycol) (PAA-g-PEG), poly(L-glutamic acid)-grafted-poly(ethylene glycol) (PGA-g-PEG), poly(sodium 4-styrenesulfonate) (PSS), dermatan sulfate, carboxymethyl cellulose (CMC), or any combinations thereof. Such scaffold optionally can be used in the form of a salt, e.g., sodium salt, lithium salt, or other similar salt. The scaffold used can be a sole scaffold, or can be a mixture of different types of scaffold. In certain embodiments, the scaffold comprises chondroitin sulfate. Chondroitin sulfate (FIG. 2) is a sulfated glycosaminoglycan (GAG) composed of a chain of alternating sugars (N-acetylgalactosamine and glucuronic acid), and usually found attached to proteins as part of a proteoglycan. A chondroitin chain can have over 100 individual sugars, each of which can be sulfated in variable positions and quantities. In certain embodiments, the chondroitin comprises about 38 repeat units (n˜38).

In certain embodiments, a polymerizable crosslinker is useful within the present invention. In other embodiments, the crosslinker comprises at least one oxiranyl

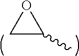

group and an alkenyl (C=C) group. In yet other embodiments, the alkenyl group is capable of undergoing radical-based polymerization. In yet other embodiments, the oxiranyl group is further optionally substituted. In yet other embodiments, the alkenyl group is further optionally substituted. Without wishing to be limited by any theory, the oxiranyl group in the crosslinker can react with a nucleophilic group on the chondroitin sulfate (such as but not limited to a carboxylate and/or an alcohol group), with formation of a new chemical bond and concomitant opening of the oxiranyl group. Without wishing to be limited by any theory, the alkenyl group of the crosslinker can undergo polymerization with alkenyl groups of other crosslinkers, forming crosslinks between chains of the scaffold. In certain embodiments, the number of alkenyl groups in the composition can be controlled; in other embodiments, the molar level can vary from about 0.1 to about 0.55. Higher crosslinking level gives a more viscous gel, with almost no flow at the highest level.

In certain embodiments, the crosslinker comprises a glycidyl group. In other embodiments, the crosslinker comprises at least one selected from the group consisting of a glycidyl ester, amide, amine, ether, thioester, sulfonamide, and any combinations thereof. In yet other embodiments, the crosslinker comprises at least one selected from the group consisting of glycidyl methacrylate (GMA), glycidyl acrylate, ally glycidyl ether, and any combinations thereof.

The invention contemplates that compositions of the invention comprise any mole or weight ratios between the scaffold and the polymerizable crosslinker, whereby upon polymerization of the composition a hydrogel is formed. In certain embodiments, varying the mole or weight ratios between the scaffold and the polymerizable crosslinker in the composition allows for the preparation of a polymerized hydrogel with tunable maximum water content and mechanical properties. In other embodiments, the polymerized hydrogel has approximately the same mechanical properties of the nucleus pulposus of a subject.

In certain embodiments, a polymerization initiator is useful within the invention. The initiator generates radical species, which promote the crosslinking of at least a portion of the alkenyl groups in the derivatized scaffold. In certain embodiments, the initiator comprises at least one selected from the group consisting of radiation (such as, but not limited to, UV and/or visible light), a chemical agent, and any combinations thereof. In other embodiments, the chemical agent is capable of generating radical species in solution. In yet other embodiments, the chemical agent is soluble in water or in aqueous solution.

In certain embodiments, the chemical agent comprises an inorganic peroxide, azo compound, or organic peroxide. Non-limiting examples of inorganic peroxides include, but are limited to, persulfuric acid or a salt thereof, hydroxymethanesulfinic acid or a salt thereof, and hydrogen peroxide. Non-limiting examples of azo compounds include, but are limited to, 4,4′-azobis(4-cyanovaleric acid), 1,1′-azobis(cyclohexanecarbonitrile), azobisisobutyronitrile, 2,2′-azobis(2-methylpropionamidine), and 2,2′-azobis(2-methylpropionitrile). Non-limiting examples of organic peroxides include tert-butyl hydroperoxide, tert-butyl peracetate, cumene hydroperoxide, 2,5-di(tert-butylperoxy)-2,5-dimethyl-3-hexyne, 2,5-di(tert-butylperoxy)-2,5-dimethyl-3-hexyne, dicumyl peroxide, 2,5-bis(tert-butylperoxy)-2,5-dimethylhexane, 2,4-pentanedione peroxide, 1,1-bis(tert-butylperoxy)-3,3,5-trimethylcyclohexane, 1,1-bis(tert-butylperoxy)cyclohexane, 1,1-bis(tert-amylperoxy)cyclohexane, benzoyl peroxide, 2-butanone peroxide solution, tert-butyl peroxide, lauroyl peroxide, tert-butyl peroxybenzoate, tert-butylperoxy 2-ethylhexyl carbonate, and tert-butyl hydroperoxide. In yet other examples, the chemical agent is provided as a net solid or liquid. In yet other examples, the chemical agent is provided as a water-free solution or an aqueous solution.

Without wishing to be limited by any theory, the use of a chemical agent to promote polymerization avoids problems associated with radiation-initiated polymerization, such as the need to prepare small batches of material at a time because radiation is unable to penetrate large amounts of material. In certain embodiments, the use of a chemical agent to polymerize the compositions of the invention allows the generation of large portions of hydrogel, which are needed for replacement of nucleus pulposus.

The hydrogels of the present invention may be further stabilized and enhanced through the addition of one or more enhancing agents. The term “enhancing agent” or “stabilizing agent” refers to any compound added to the hydrogel scaffold, in addition to the high molecular weight components, that enhances the hydrogel scaffold by providing further stability or functional advantages. The enhancing agent may include any compound, such as polar compounds, that enhance the hydrogel scaffold by providing further stability or functional advantages when incorporated in the cross-linked hydrogel scaffold. Contemplated enhancing agents for use with the stabilized cross-linked hydrogel scaffold include polar amino acids, amino acid analogues, amino acid derivatives, intact collagen, and divalent cation chelators, such as ethylenediaminetetraacetic acid (EDTA) or salts thereof. Polar amino acids include tyrosine, cysteine, serine, threonine, asparagine, glutamine, aspartic acid, glutamic acid, arginine, lysine, or histidine. In certain embodiments, the contemplated polar amino acids are L-cysteine, L-glutamic acid, L-lysine, and L-arginine. Polar amino acids, EDTA, and mixtures thereof, are also contemplated enhancing agents. The enhancing agents may be added to the scaffold composition before or during the crosslinking of the high molecular weight components. The hydrogel scaffold may exhibit an intrinsic bioactivity, which may be a function of the unique stereochemistry of the cross-linked macromolecules in the presence of the enhancing and strengthening polar amino acids, as well as other enhancing agents.

Association of the therapeutic agent with the scaffold may be accomplished via a protease sensitive linker or other biodegradable linker. Molecules that can be incorporated into the hydrogel scaffold include, but are not limited to, vitamins and other nutritional supplements; glycoproteins (e.g., collagen); fibronectin; peptides and proteins; carbohydrates (both simple and/or complex); proteoglycans; antigens; oligonucleotides (sense and/or antisense DNA and/or RNA); antibodies (for example, to infectious agents, tumors, drugs or hormones); and gene therapy reagents.

### Methods

In some instances, the present invention includes administering compositions of the invention into the nucleus pulposus of a degenerated disc for the purpose of increasing the osmotic potential of the disc, thus restoring disc height and function. In certain embodiments, the osmotic pressure of the compositions added increases the overall osmotic potential of the nucleus material. Preferably, the osmotic pressure of the composition is low enough that the resultant increase in pressure does not in itself cause pain. It is desirable to increase the osmotic pressure of the disc, because this can restore disc height and function is encompassed in the invention.

The compositions of the invention are useful for treatment of the spine, in particular, for functional restoration of the disc in the spine. The intervertebral disc comprises three major components: the nucleus pulposus, the annulus fibrosus, and a pair of cartilaginous endplates. In certain embodiments, the present invention may be practiced upon any of these sites, alone or in any combination.

The compositions and methods of the present invention can be used to treat individuals suffering from degenerated intervertebral disc conditions, in certain embodiments through restoration of osmotic potential in the intervertebral disc. By administering a composition of the invention into the intervertebral space of a degenerated disc, the damaged tissue can effectively be repaired.

The present invention provides less invasive procedures than those of the prior art for treatment of intervertebral disc disorders. In addition, the compositions and methods of the present invention can prompt biological repair of normal tissue in the disc, which results in better long term results than those obtained with synthetic prostheses. Administration of compositions of the present invention into the degenerated disc can restore normal disc height and function. For example, the compositions of the present invention can assist in the restoration of the load-bearing and viscoelastic properties of the defective intervertebral disc. The present invention can be used in conjunction with any known or heretofore unknown method of treating a disc disease or condition in a mammal, preferably a human. For example, the compositions of the present invention can be added to an adjuvant for fusion or be used in total disc arthroplasty (TDA) in adjacent discs. In addition, the compositions of the present invention can be used in adjacent discs after vertebroplasty due to compression fracture. In addition, the compositions of the present invention can be used for reconstruction in spondylolishesis or scoliosis.

The present invention includes administering compositions of the present invention to a degenerative disc to restore at least the physical element of the disc. Other components that are useful for the invention include, but are not limited to hyaluronan, chondroitin sulfate, keratan sulfate, albumin, elastin, fibrin, fibronectin, and casein.

Preferably, the nucleus pulposus portion of the intervertebral disc is selected as the target site for the administration of compositions of the present invention. Treating the nucleus pulposus with compositions of the present invention can stiffen the nucleus pulposus (thereby reducing undesired mobility).

In some embodiments, both the nucleus pulposus and the annulus fibrosis may be treated with the same administration of compositions of the present invention. In other embodiments, only the annulus fibrosis is treated.

In certain embodiments, a non-enzymatic polysaccharide oxidizing agent is injected in combination with compositions of the present invention into the nucleus pulposus of a pathological intervertebral disc. Because the dry weight component of the nucleus pulposus is rich in proteoglycans, there are numerous sites that can be oxidized to form functional aldehydes. Subsequently, the aldehydes can react with amino acid regions of both native and non-native collagens and proteoglycans to form a network of molecules.

In another aspect, compositions of the present invention are attached to a polymer backbone such as polyethylene glycol or polyvinyl alcohol or other HA (hyalouronic acid) analog. The backbone is used to implant compositions of the present invention into the intervertebral disc. The backbone is also useful for providing structure to prevent compositions of the present invention from migrating out of the intervertebral disc (e.g., the nucleus space).

The invention also includes the use of viable cells in combination with compositions of the present invention. Examples of such cells include harvested cells selected from the group consisting of healthy nucleus pulposus or annulus fibrosus cells, precursors of nucleus pulposus or annulus fibrosus cells, or cells capable of differentiating into nucleus pulposus or annulus fibrosus cells. In some instances, compositions of the present invention can be used as a cell matrix for supporting both in vivo as well as in vitro cell culture.

Also included in the invention is a hybrid material in which cells are combined with compositions of the present invention. Intervertebral disc cells may be isolated from tissue extracted from any accessible intervertebral disc of the spine. For example, tissue may be extracted from the nucleus pulposus of lumbar discs, sacral discs or cervical discs. Preferably, intervertebral disc cells are primarily nucleus pulposus cells. In some embodiments, it is preferred that disc cells are at least 50% nucleus pulposus cells while 90% nucleus pulposus cells is still more preferred. Cells may be obtained from the patient being treated, or alternatively cells may be extracted from donor tissue. Methods to isolate and culture disc cells including but not limited to precursor and/or nucleus pulposus cells are described in U.S. Application Publication No. US 2013/0052155, all of which is incorporated herein in its entirety.

In the event that intervertebral disc cells are not available, the invention includes the use of any cell that is capable of differentiating into a disc cell, such as stem cells, which include, but are not limited to embryonic stem cells and adult stem cells derived or obtained from any source, preferably a human source.

In another aspect of the invention, the desired cells may be allogeneic with respect to the recipient. The allogeneic cells are isolated from a donor that is a different individual of the same species as the recipient. Following isolation, the cells are cultured using standard culturing methods to produce an allogeneic product. The invention also encompasses cells that are xenogeneic with respect to the recipient.

Following isolation, the cells of the invention are incubated in the desired cell medium in a culture apparatus for a period of time or until the cells reach confluency before passing the cells to another culture apparatus. The culturing apparatus can be any culture apparatus commonly used in culturing cells in vitro. Preferably, the level of confluence of the cells is greater than 70% before transferring the cells to another culture apparatus. More preferably, the level of confluence is greater than 90%. A period of time can be any time suitable for the culture of cells in vitro. Cell medium may be replaced during the culture of the cells at any time. Preferably, the cell medium is replaced every 2 to 4 days. Cells are then harvested from the culture apparatus whereupon the cells can be used immediately or cryopreserved and stored for use at a later time. Cells may be harvested using trypsinization, EDTA treatment, or any other procedure used to harvest cells from a culture apparatus.

### Administration

The present invention provides a method for restoring a damaged or degenerated intervertebral disc comprising administering administerable compositions of the present invention. The administerable composition can either be viscous or form a solid or gel in situ.

The compositions of the present invention may be administered to a soft tissue site in a vertebrate, for the functional restoration thereof, using a variety of methods and in a variety of formulations known in the art. The vertebrate may be a mammal, and the mammal may be a human.

In some instances, it is preferable that the composition of the invention does not appreciably degrade following administration. In other instances, it is preferred that the composition of the invention degrades either rapidly, or slowly, in the tissue. Thus, when administered in the body, compositions of the present invention may be permanent, may be degraded enzymatically, or may be degraded in the presence of a solvent, such as, for example, water.

When administered to a disc, and recognizing that the methods and formulations disclosed herein are equally applicable to other tissues, it is envisioned that any suitable annular closure technique may be used before or after insertion of compositions of the present invention into the disc tissue. The annular closure technique can be applied before or after administration. Examples of suitable closure techniques may include the use of the following alone or in combination, sutures (resorbable or non- resorbable strips/cords/draw strings/wires/cords), adhesives (fibrin, cyanoacrylates, polyanhydrides, glutaraldehydes, platelet-rich plasma and so forth), in-situ fabricated plugs (single sheet wound or two piece snapped together), pre-fabricated plugs (like a tire plug), expandable plugs (stent like), for example.

Delivery of the desired material into the nucleus pulposus or annulus fibrosus of the disc may be by delivery through the ruptured area of the annulus, by delivery through a separate passageway way through or into the annulus, or by delivery through a plug or other closure device used to repair the ruptured annulus. Delivery of the material can also be accomplished by direct administration into the nucleus pulposus.

In certain embodiments, the compositions are nonaqueous (does not contain water) and solid or gel forming (turns into a solid or gel in situ).

In certain embodiments, the compositions are nonaqueous and comprise an organic solvent or a mixture of organic solvents. Metabolically absorbable solvents are preferably selected (triacetin, ethyl acetate, ethyl laurate, and the like).

In certain embodiments, the compositions are nonaqueous and contain at least one fatty acid or a mixture of fatty acids. The compositions may comprise saturated or unsaturated fatty acid selected from the group consisting of palmitate, stearate, myristate, palmitoleate, oleate, vaccenate and linoleate. They may be a mixture of several of these fatty acids. The fatty acid may be mixed with a metabolically absorbable solvent or liquid vehicle to reduce viscosity and allow administerability.

In certain embodiments, the compositions are a dry powder, which when introduced into the soft tissue, e.g., the disc, is hydrated within the tissue to result in the desired restoration thereof.

In certain embodiments, the compositions of the invention are dried gels, wherein water is mostly or completely removed by drying in a vacuum oven or by dialysis.

In certain embodiments, the compositions of the invention may be injected directly into the degraded human nucleus pulposus in the intervertebral disc of a patient. In other embodiments, the dry gel gradually absorbs water from the body and grows in volume, thereby restoring the hydrostatic pressure, volume, and mechanical properties of the degraded nucleus pulposus.

An advantage of the present invention is that the entire intervertebral disc is not removed in order to effect treatment of the degenerated disc. However, it is recognized that in some instances, the materials of the present invention can be administered into the degenerated disc without removing native material from the degenerated disc prior to administration of the materials. The purpose of removing native material from the degenerated disc is to make room for the materials to be administered.

When cells are used to treat a degenerated disc, the cells may be administered to a mammal following a period of in vitro culturing. The cell may be cultured in a manner that induces the cell to differentiate in vitro. However, the cells can be administered into the recipient in an undifferentiated state where the administered cells differentiate to express at least one characteristic of a disc cell in vivo in the mammal

The cells of this invention can be transplanted into a mammal using techniques known in the art such as i.e., those described in U.S. Pat. No 5,618,531, all of which is incorporated herein by reference, or into any other suitable site in the body. Transplantation of the cells of the present invention can be accomplished using techniques well known in the art as well as those described herein, or using techniques developed in the future. The present invention comprises a method for transplanting, grafting, infusing, or otherwise introducing the cells into a mammal, preferably, a human.

The cells can be suspended in an appropriate diluent. Suitable excipients for administration solutions are those that are biologically and physiologically compatible with the cells and with the recipient, such as buffered saline solution or other suitable excipients. The composition for administration can be formulated, produced and stored according to standard methods complying with proper sterility and stability.

The cells may also be encapsulated and used to deliver biologically active molecules, according to known encapsulation technologies, including microencapsulation (see, e.g., U.S. Pat. Nos. 4,352,883; 4,353,888; and 5,084,350, all of which are herein incorporated by reference), or macroencapsulation (see, e.g., U.S. Pat. Nos. 5,284,761; 5,158,881; 4,976,859; and 4,968,733; and International Publication Nos. WO 92/19195; WO 95/05452, all of which are incorporated herein by reference). For macroencapsulation, the number of cells used in the devices can be varied. Several macroencapsulation devices may be administered in the mammal Methods for macroencapsulation and administration of cells are well known in the art and are described in, for example, U.S. Pat. No. 6,498,018.

The mode of administration of the cells of the invention to the mammal may vary depending on several factors including the type of disease being treated, the age of the mammal, whether the cells are differentiated or not, whether the cells have exogenous DNA introduced therein, and the like. The cells may be introduced to the desired site by direct administration, or by any other means used in the art for the introduction of compounds administered to a mammal suffering from a particular disease or disorder of the disc.

The invention further provides, in some aspects, methods of treating a degenerative disc by administering a composition comprising at least one selected from the group consisting of a cell, a matrix, a cell lysate, a cell-product of the invention (i.e. molecules secreted by the cell), and any combinations thereof in a mammal in need thereof. As such, the invention encompasses a pharmaceutical composition, wherein the composition may be used in the treatment of a bone condition such as a degenerated disc.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

The following examples further illustrate aspects of the present invention. However, they are in no way a limitation of the teachings or disclosure of the present invention as set forth herein.

### EXPERIMENTAL EXAMPLES

The invention is now described with reference to the following Examples. These Examples are provided for the purpose of illustration only, and the invention is not limited to these Examples, but rather encompasses all variations that are evident as a result of the teachings provided herein.

### Example 1

### Physical Characterization

Compositions comprising varying ratios of chondroitin sulfate (CS) and glycidyl methacrylate (GMA) were prepared, crosslinked and polymerized. The resulting hydrogels were physically characterized.

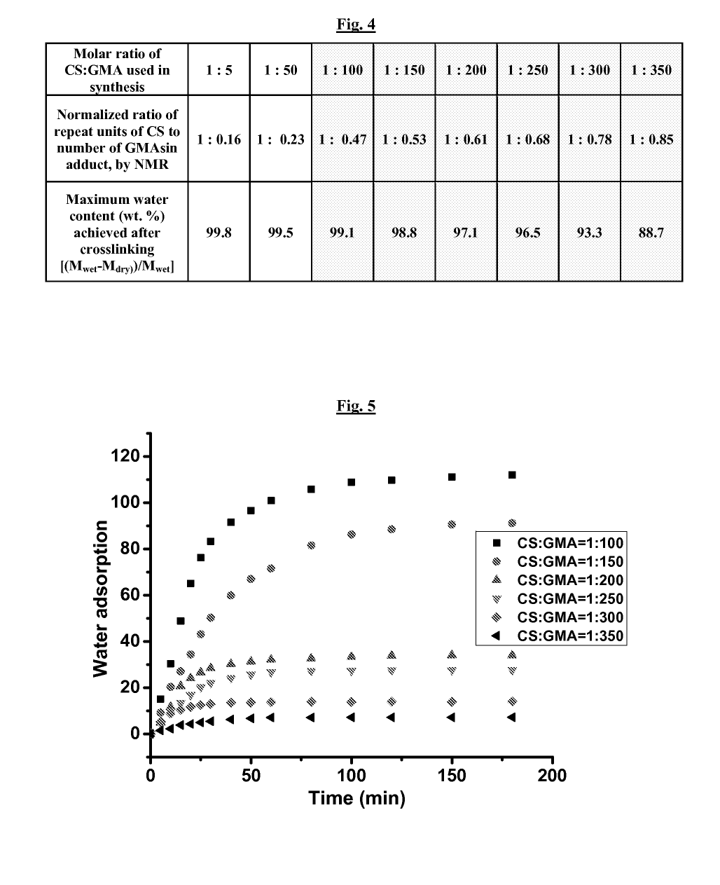

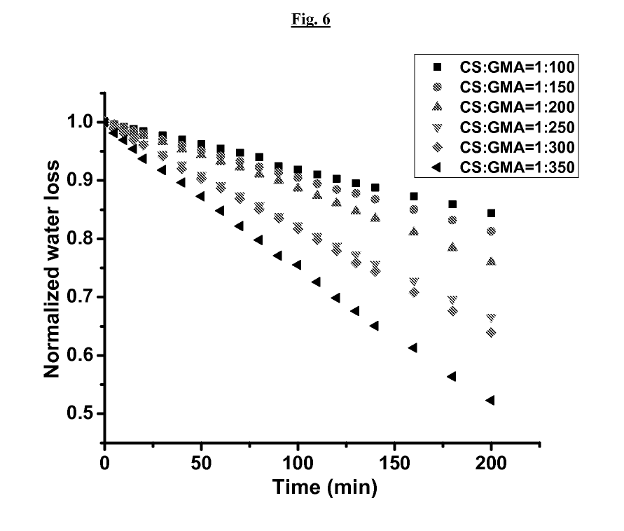

The table in FIG. 4 summarizes the results of the experiments, wherein the molar amount of GMA relative to the molar mount of CS was increased from left to right. Nuclear magnetic resonance studies showed that, as relative amount of GMA was increased, the number of GMA molecules chemically bonded to the CS chain increased. While crosslink density (per volume) was not directly measured, higher ratios of GMA to CS produced higher crosslink densities, as indicated by measurements of maximum water absorption for each formulation after crosslinking was carried out.

Maximum water absorption is illustrated in the bottom row of FIG. 4, left to right. A higher maximum water absorption was associated with a lower crosslink density, consistent with a higher fraction of free water (and a lower fraction of bound water) for lower crosslink densities. The water absorption capabilities spanned by the hydrogel of the invention include the range of absorbed water values (70-90% by weight) described for the human nucleus pulposus.

FIG. 5 illustrates the water absorption properties of hydrogels of the invention.

The absorption ratio was calculated as the ratio of mass of water in sample and mass of dry polymer.

FIG. 6 illustrates the water loss for hydrogels of the invention. Normalized water loss was calculated as the ratio between the mass of wet sample and the mass of wet sample with maximum water content.

### Example 2

### Mechanical Properties

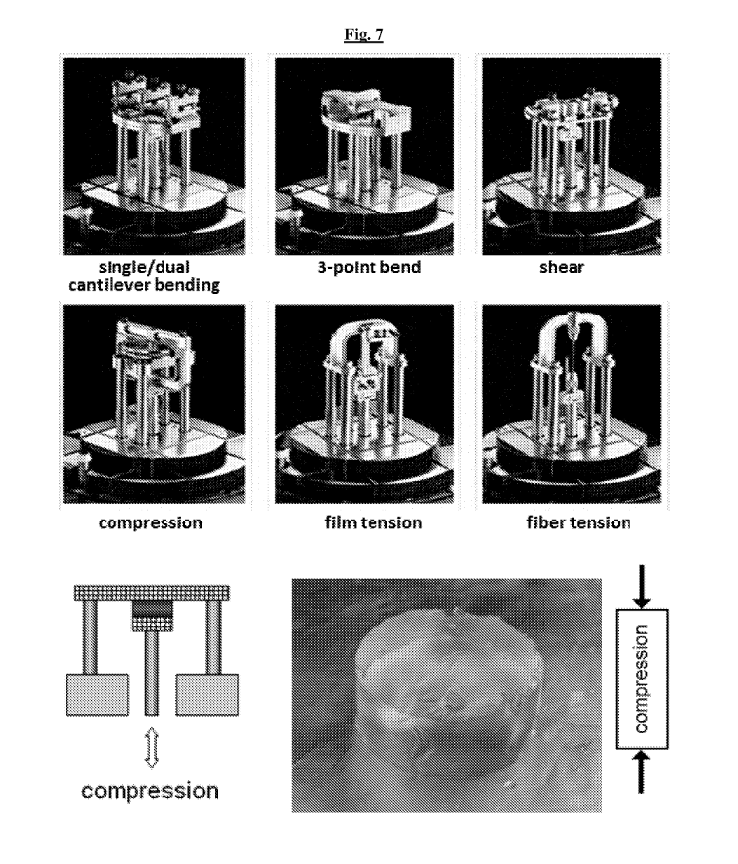

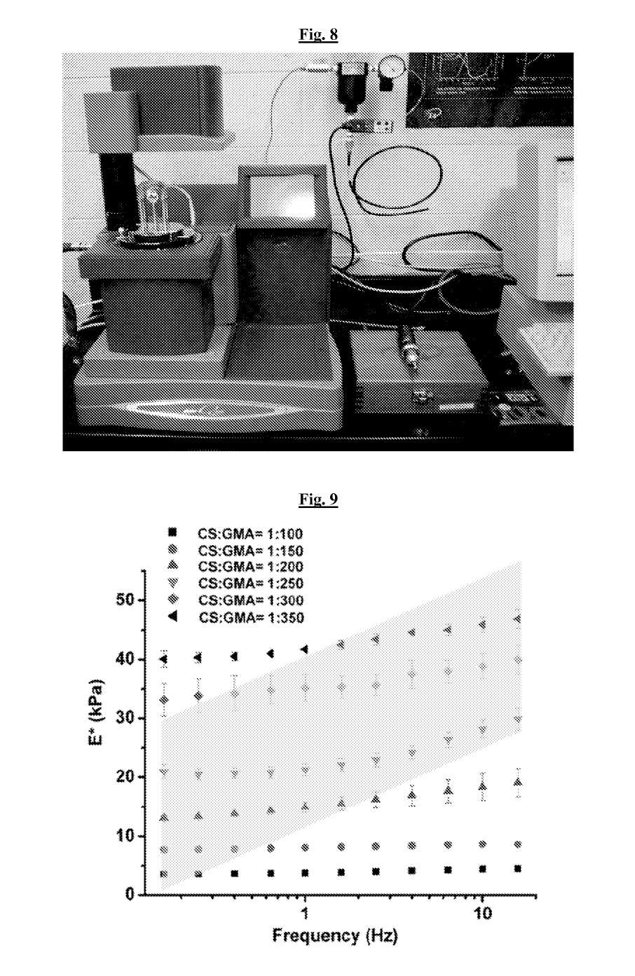

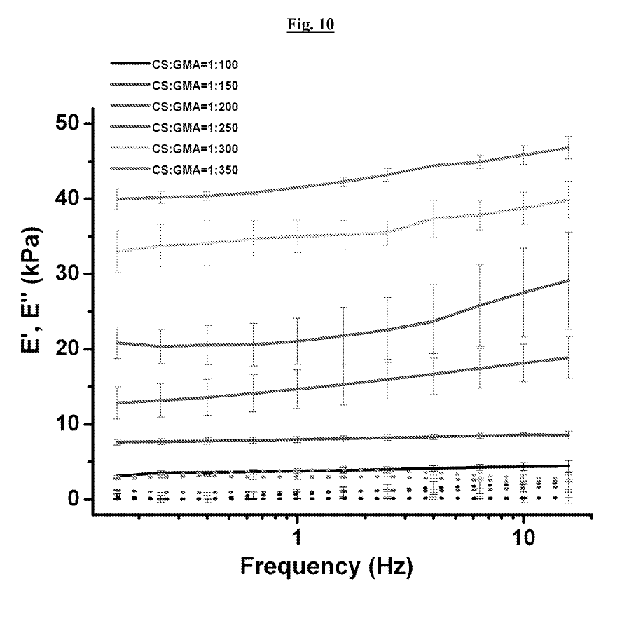

Materials in general respond to variable stress, temperature or frequency (FIG. 7). Dynamic mechanical analysis (DMA, illustrates in FIG. 8) measures small deformations applied to materials. DMA allows for the characterization of viscoelastic properties of materials. Storage modulus (E′ ) corresponds to the energy stored elastically during deformation, and relates to elastic modulus of solids. Loss modulus (E″ ) corresponds to the portion of the energy delivered to the material that is not recovered, i.e., that is dissipated as heat, and relates to viscous behavior. The complex dynamic modulus (E*=E′+iE″ ) is a measure of stiffness during dynamic loading. The phase angle δ is a measure of dissipation during dynamic loading: δ=tan⁻¹ [E″/E′].

Hydrogels of the invention were characterized for storage modulus (E′ ) and loss modulus (E″ ). The materials were submitted to compression mode at room temperature (constant temperature), and the applied strain was cyclic at maximum of 0.76% with preload of 65 Pa. The frequency was varied (0.16 Hz at 16 Hz).

Dynamic mechanical test results from torsion (shear) loading can be converted to the corresponding results from normal (tension or compression). This allowed the conversion of cyclic shear values into equivalent cyclic compression values.

FIG. 9 illustrates data for complex compression modulus (E*), and the shaded area corresponds to the equivalent compression values derived from literature studies on normal (not degenerated) nucleus pulposus. The hydrogel formulations provided values of complex modulus that span a large portion of the range of values associated with human nucleus pulposus. In fact, studies on human nucleus pulposus show great data scatter, with recited values of E*=14±15 kPa at 1.6 Hz. In certain embodiments, compositions of the invention including lower ratios of chondroitin sulfate to crosslinker (such as 1:300 or 1: 350, for example) provide hydrogels with higher values of complex modulus.

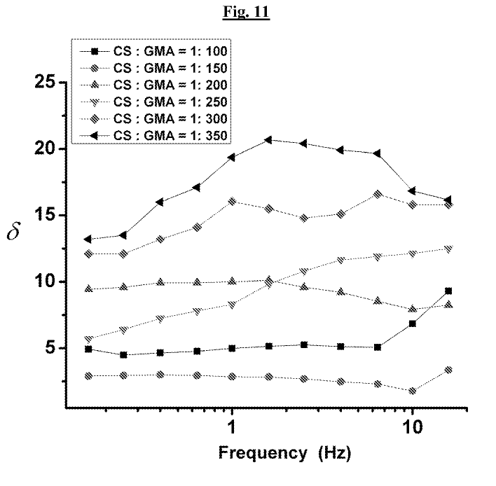

As illustrated in FIG. 10, experiments with hydrogels of the invention indicated that the storage modulus E′ was much larger than the loss modulus E″ and the phase angle is <45 degrees for all formulations. This establishes that the hydrogels of the invention, like the naturally occurring nucleus pulposus, is a viscoelastic solid (and not a fluid).

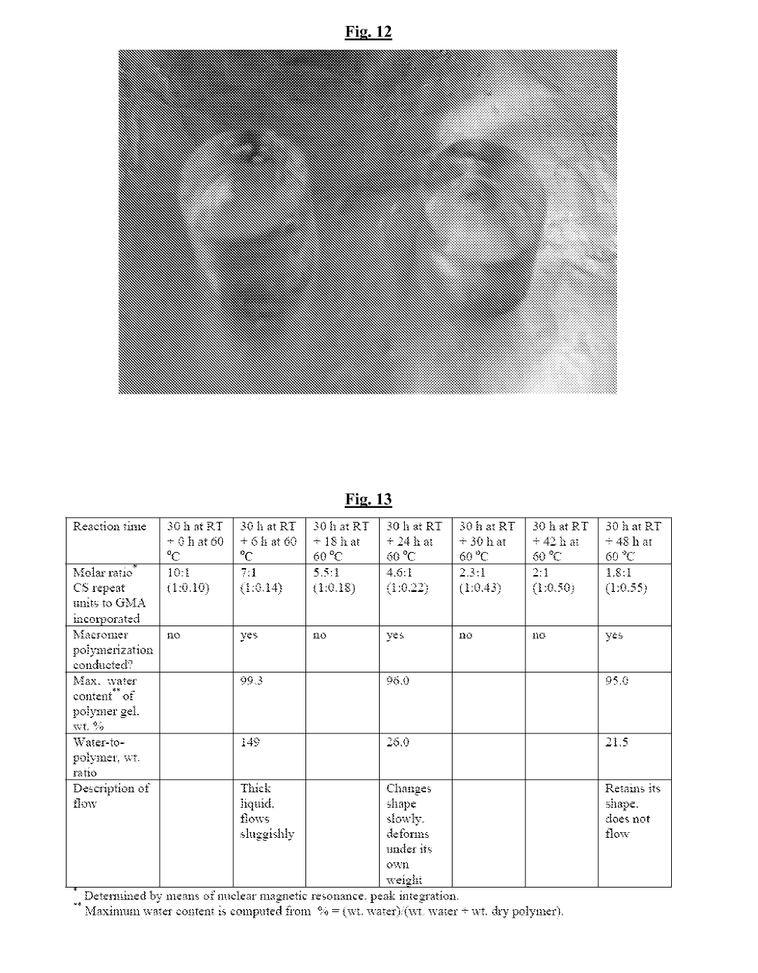

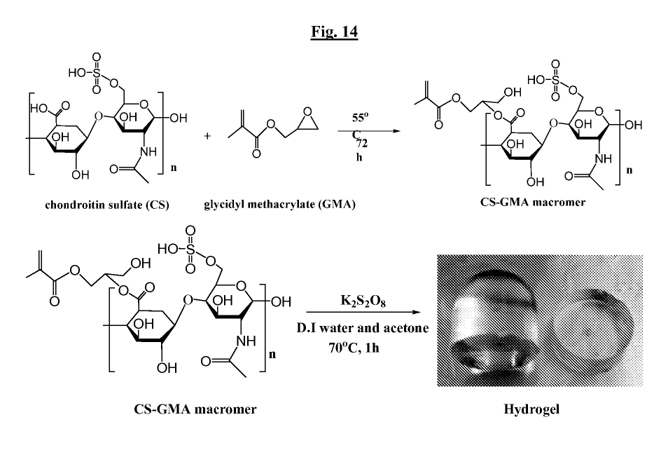

As illustrated in FIG. 11, hydrogels of the invention provide phase angles that are consistent with viscoelastic solids.

As illustrated in FIG. 12, the hydrogels of the invention display the shape retention behavior of a viscoelastic solid.

In certain embodiments, the hydrogels of the invention are a suitable replacement or supplement for the human nucleus pulposus. The complex modulus of hydrogels of the invention can mimic that of nucleus pulposus. In certain embodiments, the nucleus pulposus has E* of 12-32 kPa, and the hydrogels of the invention have E* of 3-45 kPa over a range of frequency. In other embodiments, the hydrogels of the invention have similar water absorption capability (to a maximum of 96-99%) to that of nucleus pulposus (85-90% in healthy nucleus pulposus).

Further, the hydrogels of the invention are biocompatible. In certain embodiments, the hydrogels are composed of chondroitin sulfate, a biopolymer found in the nucleus pulposus. In other embodiments, non-biological molecules are mostly or totally extracted in the work-up, so that the hydrogels contain no redox initiator and no unreacted glycidyl methacrylate.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A composition comprising a polymerizable crosslinker and a scaffold,
wherein the polymerizable crosslinker comprises an oxiranyl group and an alkenyl group, and
wherein the scaffold comprises at least one selected from the group consisting of collagen, pectin, carrageenan, poly(L-lysine), gelatin, agarose, dextran sulfate, heparin, polygalacturonic acid, mucin, chondroitin sulfate, hyaluronic acid, chitosan, alginate, alginate sulfate, poly(acrylic acid), poly(methyl methacrylate) (PMMA), poly(vinylsulfonic acid) (PVSA), poly(L-aspartic acid), poly(L-glutamic acid), poly(L-aspartic acid)-grafted-poly(ethylene glycol) (PAA-g-PEG), poly(L-glutamic acid)-grafted-poly(ethylene glycol) (PGA-g-PEG), poly(sodium 4-styrenesulfonate) (PSS), dermatan sulfate, carboxymethyl cellulose (CMC), and any combinations thereof.

2. The composition of claim 1, wherein the scaffold comprises chondroitin sulfate.

3. The composition of claim 1, wherein the crosslinker comprises a glycidyl group.

4. The composition of claim 3, wherein the crosslinker comprises at least one selected from the group consisting of a glycidyl ester, amide, amine, ether, thioester, sulfonamide, and any combinations thereof.

5. The composition of claim 4, wherein the crosslinker comprises at least one selected from the group consisting of glycidyl methacrylate (GMA), glycidyl acrylate, allyl glycidyl ether, and any combinations thereof.

6. The composition of claim 1, wherein the scaffold and polymerizable crosslinker react to form a derivatized scaffold.

7. The composition of claim 6, wherein the ratio between the scaffold and the polymerizable crosslinker is selected so that at least partial crosslinking of the derivatized scaffold generates a hydrogel.

8. The composition of claim 1, wherein the composition further comprises a polymerization initiator comprising a chemical agent.

9. The composition of claim 8, wherein the chemical agent comprises at least one selected from the group consisting of an inorganic peroxide, azo compound, organic peroxide, and any combinations thereof.

10. The composition of claim 8, wherein the polymerization initiator is at least partially soluble in the composition.

11. The composition of claim 6, wherein at least a portion of the derivatized scaffold is crosslinked.

12. The composition of claim 11, wherein crosslinking of the scaffold is promoted by contacting the derivatized scaffold with a polymerization initiator comprising a chemical agent.

13. The composition of claim 12, wherein the composition comprises a hydrogel.

14. The composition of claim 13, wherein the hydrogel has about the same mechanical properties of the nucleus pulposus of a subject.

15-19. (canceled)

20. A method of preparing a hydrogel, the method comprising contacting a scaffold and a polymerizable crosslinker to generate a derivatized scaffold, and promoting at least partial crosslinking of the derivatized scaffold, whereby a hydrogel is formed,
wherein the polymerizable crosslinker comprises an oxiranyl group and an alkenyl group, and
wherein the scaffold comprises at least one selected from the group consisting of collagen, pectin, carrageenan, poly(L-lysine), gelatin, agarose, dextran sulfate, heparin, polygalacturonic acid, mucin, chondroitin sulfate, hyaluronic acid, chitosan, alginate, alginate sulfate, poly(acrylic acid), poly(methyl methacrylate) (PMMA), poly(vinylsulfonic acid) (PVSA), poly(L-aspartic acid), poly(L-glutamic acid), poly(L-aspartic acid)-grafted-poly(ethylene glycol) (PAA-g-PEG), poly(L-glutamic acid)-grafted-poly(ethylene glycol) (PGA-g-PEG), poly(sodium 4-styrenesulfonate) (PSS), dermatan sulfate, carboxymethyl cellulose (CMC), and any combinations thereof.

21. The method of claim 20, wherein the scaffold comprises chondroitin sulfate.

22. The method of claim 20, wherein the polymerizable crosslinker comprises glycidyl methacrylate.

23. The method of claim 20, wherein promoting at least partial crosslinking of the derivatized scaffold comprises contacting the derivatized scaffold with a polymerization initiator comprising a chemical agent.

24. The method of claim 20, wherein the hydrogel has about the same mechanical properties of the nucleus pulposus of a subject.

25. A method of replacing or supplementing the nucleus pulposus in a subject in need thereof, the method comprising administering to the subject a therapeutically effective amount of the hydrogel of claim 13.

26-30. (canceled)
